(19) United States
(12) Patent Application Publication
(10) Pub. No.: US 2025/0001193 A1
(43) Pub. Date: Jan. 2, 2025

(54) STIMULATION ARRANGEMENT AND METHOD OF IDENTIFYING A POSITION OF A FIELD GENERATOR

(71) Applicant: STIMIT AG, Biel/Bienne (CH)

(72) Inventor: Ronja MÜLLER-BRUHN, Windisch (CH)

(21) Appl. No.: 18/710,468

(22) PCT Filed: Nov. 23, 2022
(86) PCT No.: PCT/EP2022/082903

§ 371 Date: May 15, 2024

(30) Foreign Application Priority Data
Nov. 24, 2021 (CH) 070594/2021

(51) Int. Cl. A61N 2/02; A61N 2/00

(52) U.S. Cl. CPC A61N 2/02; A61N 2/006

(57) ABSTRACT

A stimulation arrangement (10) comprises a field generator (21) configured to generate a spatial field, and a control unit (3) in communication with the field generator. The field generator (21) is configured to be positioned at a human or animal patient (5) such that a muscular structure of the patient (5) is activatable by the spatial field. The control unit (3) is configured to control the field generator (21) to generate the spatial field. The control unit (3) is configured to operate the field generator (21) to generate a positioning field having an increasing intensity.

### TECHNICAL FIELD

The present invention relates to a stimulation arrangement according to the preamble of independent claim 1 and more particularly to a method of identifying a position of a field generator.

Such stimulation arrangement having a field generator configured to generate a spatial field, and a control unit in communication with the field generator and configured to control the field generator to generate a spatial field, can be used to be positioned at a human or animal patient in order to stimulate a muscular structure of the patient by means of the spatial field. For example, such stimulation arrangements can be used to stimulate an aspiration muscular structure such as a diaphragm via the phrenic nerves for ventilating the patient.

### BACKGROUND ART

In medicine, it is known that for many purposes it is beneficial to activate a target tissue of a patient. Such activation may be aimed for therapeutic purposes, wherein typically a muscular structure is activated to achieve the intended therapeutic effect.

For example, in critical care units of hospitals it may be desired to activate the diaphragm of ventilated patients in order to prevent drawbacks of disuse of the diaphragm. It was shown that disuse atrophy of diaphragm muscle fibers occurs already in the first 18-69 hours of mechanical ventilation, and the muscle fiber cross-sections decreased by more than 50% in this time. Thus, it is aimed to activate the diaphragm repeatedly while the patient is given artificially or mechanical respiration such that the functioning of the diaphragm can be upheld, or to activate the diaphragm at least during the weaning period to support effective restoration of independent respiratory function.

Or, in another example, in hospitals or other care units it is desired to ventilate patients in therapeutic treatments. Conventionally, such ventilation is provided by imposing mechanical positive pressure air into the respiratory system of the patient. Alternatively, it is known to activate the respiratory muscular system of the patient in order to move (pump) air into the lungs via negative pressure.

For achieving such activation of tissues and, particularly, muscular structures in a patient's body, it is known to directly stimulate the tissue or to indirectly activate the tissue via stimulation of specific parts of the neural system. For example, the target tissue being a muscular tissue can be activated by providing electric pulses directly to the tissue or to nerves associated to the tissue. More specifically, it is known that the diaphragm can be activated by stimulating the phrenic nerve, e.g., at the neck of the patient.

In this context, US 2016/0310730 A1 describes an apparatus for reducing ventilation induced diaphragm disuse in a patient receiving ventilation support from a mechanical ventilator (MV). The apparatus includes an electrode array of first and second types and comprising a plurality of electrodes configured to stimulate a phrenic nerve of the patient, and at least one controller identifying a type of electrode array from at least two different types, and generating a stimulus signal for stimulating a phrenic nerve of the patient based upon the identity of the electrode type. Such electrode-based stimulation is not very robust to patient movements or relocations, and the possible stimulation depth can be significantly limited by bones or fatty tissue. Furthermore, electrode stimulation is reported to be more painful for the patient than electro-magnetic stimulation.

Further, WO 2019/154837 A1 describes an electro-magnetic induction device for stimulating a phrenic nerve of a patient by means of a spatial electro-magnetic field applied on the neck of the patient. The device comprises an electro-magnetic field generator with a coil design configured to generate the electro-magnetic field with a targeted shape. It further has a sensor member configured to detect an activation of the target tissue and an electro-magnetic field adjustment mechanism configured to automatically adjust a position of the electro-magnetic field generated by the coil design. For finding an appropriate position of the electro-magnetic field generator such that the phrenic nerve can be efficiently stimulated, the device is equipped with a calibration unit which is in communication with the sensor member and with the electro-magnetic field adjustment mechanism. The calibration unit of the electro-magnetic induction device according to the invention is configured to automatically vary the position of the electro-magnetic field generated by the coil design, and to automatically stop variation of the position of the electro-magnetic field generated by the coil design when an activation feedback signal is received from the sensor member.

Event though the known device allows for an efficient and convenient positioning of the electro-magnetic field generator, it has drawbacks as it requires comparably complex structures for automatically varying the electro magnetic field. Further, applying the electro-magnetic field for positioning the electro-magnetic field generator may affect the patient.

For example, it is known to use different impulse train forms such as “single twitch” or “continuous stimulation” or “ramp followed by plateau” to stimulate the diaphragm in order to identify diaphragmatic activity.

However, single impulses (twitches) appear sudden and create discomfort for the patient. Especially in combination with electro-magnetic fields, but also with other stimulation approaches based on electric stimulation, a single twitch creates patient discomfort because a single twitch-especially when higher stimulation intensities are required—is a short unexpected impulse and creates a feeling of “touching an electric fence” for the patient.

A simple plateau-stimulation at a “set value” of electric field/electro-magnetic field magnitude creates a tetanic diaphragm contraction. However, using such “set value” creates the same “sudden” feeling like a sudden unexpected application of impulses at high intensities is not comfortable for the patient.

Gradually increasing trains who reach a plateau an keep the magnetic field for a relatively long time on a set level will affect the breathing pattern, and thus may have undesired impact on the ventilator frequency, the breathing rate, may lead to irregularities in the breathing pattern, or may trigger a ventilator in an undesirable time window, may not be compatible with all ventilator modes. plateau-shaped rectangular trains at a “set value” must be properly synchronized to the inspiratory time window of the ventilator and/or the patient: tetanic diaphragm contractions produce significant tidal volumes, significant flow. If applied in an unfavorable time window of the breathing pattern too often (e.g. too late in inspiration or too early in expiration) the breathing patterns of the patient or ventilator may be unfavorably impacted, asynchronies may occur, patient discomfort may occur.

Typical stimulation pattern with a plateau and stimulation above threshold furthermore take too much time. They are only possible at the fastest in synchrony with each inspiration and time is lost to find the right position of the stimulation applicator (coil or electrode).

Moreover, plateau-shaped rectangular trains at a “set value”—and even more plateau-shaped trains complemented by ramps in the beginning and end produce no characteristic signal—it is extremely difficult to separate such stimulations from a natural spontaneous breath. Such separation/reliable identification of stimulated breath only works in fully sedated patients without spontaneous breath, or in patients with spontaneous breath it works only if every other breath or every third breath is stimulated when changes in breathing patterns occur.

Therefore, there is a need for a comparably simple system allowing a gentle and efficient positioning of a field generator for appropriately stimulating or activating a muscular structure.

### DISCLOSURE OF THE INVENTION

According to the invention this need is settled by a stimulation arrangement as it is defined by the features of independent claim 1, and by a method of identifying a position of a field generator at a human or animal patient as it is defined by the features of independent claim 20. Preferred embodiments are subject of the dependent claims.

In one aspect, the invention is a stimulation arrangement, which comprises a field generator and a control unit. The field generator is configured to generate a spatial field and to be positioned at a human or animal patient such that a muscular structure of the patient is activatable by the spatial field.

The control unit is in communication with the field generator. It further is configured to control the field generator to generate the spatial field and to operate the field generator to generate a positioning field having an increasing intensity.

Unless being further or differently specified in context of an embodiment or an aspect of the invention, the following definitions and explanations do apply to all embodiments and aspects of the invention.

The muscular structure of the patient can be a single muscle or a group of muscles of the patient. Advantageously, it includes an inspiration muscular structure such as a diaphragm of the patient, an external intercostal muscle of the patient, an accessory muscle of inspiration of the patient, or a combination thereof. By stimulation any of these muscular structures or even a combination thereof, aspiration can efficiently be induced. Like this, ventilation of the patient can be assisted or induced.

The term “position” as used herein refers to a location and orientation. Changing the position of an element involves either relocating the element, reorienting the element or a combination thereof. If an element or component is positioned to be capable of doing something, it advantageously is located and orientated to achieve the respective function. For example, the field generator being positioned to stimulate a phrenic nerve may relate to being located and oriented such that the phrenic nerve is within the spatial field generated by the field generator.

The term “positioned at a body” or, similarly, “holding at a body” relates correspondingly to be located and oriented at the body. In connection with the field generator these terms can relate to being physically in contact with a body of the patient or in close distance to it. The location and orientation of the field generator or a component of it can thereby be predefined or distinct to be appropriate for activating the muscular structure. In order to be configured for being positioned at an appropriate position, the field generator can be formed to be suited to the respective position. For example, it can be formed in in correspondence to a neck of the patient such that it can conveniently be positioned at the neck, e.g., for stimulating a phrenic nerve. Also, it can be equipped with an appropriate mounting structure for being held or secured at the location.

Activation of the muscular system can be directly or indirectly induced by the field generator. For example, direct activation can be induced by providing the spatial field to the muscular system or a specific muscle thereof such that the muscular system is stimulated by the spatial field. In such situations the muscular system is directly stimulated for being activated. Indirect activation can, e.g., be induced by providing the spatial field to a specific portion of the nervous system associated to the muscular system or to the specific muscle thereof such that the specific portion of the nervous system is stimulated by the spatial field. In such situations, the muscular system is activated by stimulating the nervous system. In an exemplary specific embodiment where a diaphragm is intended to be activated, e.g. for ventilation, indirect activation can be induced by positioning the field generator such that one or both phrenic nerve(s) are located in the spatial field generated by the field generator thereby stimulating the phrenic nerve(s).

For the control unit being in communication with another component such as the field generator or a sensor unit, it can be wiredly or wirelessly coupled to the other component. Like this, signals such as control signals can be transmitted to the other component for operating or controlling. Additionally or alternatively, signals such as sensor signals can be received by the control unit. For example, such sensor signals may represent a sensed dimension or physical property, e.g. for further evaluation.

The control unit can be any computing entity suitable for performing the tasks involved for controlling another component and/or for evaluating a signal such as a sensed signal. It can be or comprise a laptop computer, a desktop computer, a server computer, a tablet, a smartphone or the like. The term “control unit” covers single devices, embedded systems as well as combined devices. The control unit can, for example, be a distributed system, such as a cloud solution, performing different tasks at different locations.

Typically, control units or computers involve a processor or central processing unit (CPU), a permanent data storage having a recording media such as a hard disk, a flash memory or the like, a random access memory (RAM), a read only memory (ROM), a communication adapter such as an universal serial bus (USB) adapter, a local area network (LAN) adapter, a wireless LAN (WLAN) adapter, a Bluetooth adapter or the like, and a physical user interface such as a keyboard, a mouse, a touch screen, a screen, a microphone, a speaker or the like. Control units or computers can be embodied with a broad variety of components.

The control unit can be partially or fully embodied as separate entity, or as a part integrated in any other device or entity. For example, it can be integrated in a ventilation arrangement such as being embodied in a ventilation machine used for ventilating a human or animal patient, and/or in the induction device.

The term “spatial field” as used herein relates to any field allowing stimulation of a target tissue of the patient. It may particularly involve an electric field or an electro-magnetic field. Such spatial fields allow for directly stimulating muscular structures for activation or indirectly activating muscular structures via stimulation of the nervous system or via other muscular structures.

The spatial field can be configured to have a targeted shape. Such targeted shape can be achieved by providing a locally constrained, targeted electric or electro-magnetic field, e.g., having a peak. It can be adapted to be active in a target area being the nerve area, muscle area or tissue area that shall be stimulated with the spatial field (e.g. the phrenic nerve to be stimulated), which can be for example achieved by the peak of the spatial field (focality area). The targeted shape can generally be any shape of the spatial field or component thereof that allows to stimulate one or more target nerves, muscles or other tissue effectively while minimizing other undesired co-stimulation effects of surrounding, above-lying or close-by tissues or nerves. A peak shape is such example, because it maximizes effects in the focality area and minimizes effects outside this focality area.

For generating the spatial field, the field generator can comprise a coil design. Thereby, the term “coil design” can be or comprise at least two coils or at least one cone shaped or otherwise curved or bulged coil, or at least one cylindrical or otherwise non-flat coil, or at least one small coil, i.e. a coil sufficiently small to generate a sharp electro-magnetic field such as a coil having a diameter of 3 cm or less.

The positioning field can be provided to have different portions wherein at least one of these portions has the increasing intensity or field strength. For example, the positioning field can have a portion with a linearly increasing intensity (ramp) and a following portion with a steady intensity (plateau). Thereby, advantageously, the temporal width of the plateau is considerably smaller than the temporal width of the ramp. Also, sinus-like intensities are possible which include increasing and decreasing portions. Such ramps without a plateau or with a very short plateau only allow for producing a characteristic feedback signal change only, without producing significant flow that would impact the breathing pattern significantly (i.e. the flow threshold below trigger or tidal volume without impact on lung protection/patient comfort).

The parameters of the voltage or current waveform applied to the coil design by a generator may affect the temporal characteristics of the electromagnetic field, including pulse shape, amplitude, width, polarity, and repetition frequency; duration of and interval between bursts or trains of pulses; total number of pulses; and interval between stimulation sessions and total number of sessions have, amongst others, an influence on the field strength and determine if and with which intensity or “dose” a target area or target tissue can be stimulated.

By the control unit being configured to operate the field generator to generate a positioning field having an increasing intensity, in the invention allows for increasing comfort when setting up the stimulation arrangement to be ready for therapeutic stimulation or activation. In particular, it allows for reducing or minimizing impact on the breathing pattern, saving time, simplifying the workflow and avoiding further risks. More specifically, applying ramp-shaped comparably short pulses of the spatial field allows for conveniently identifying an appropriate or even an optimized position of the field generator. When used in connection with ventilation such identification of the appropriate position of the field generator allows to reduce or prevent significant flow that could impact the breathing pattern significantly. In particular, a flow threshold can be held below a trigger or tidal volume without impact on lung protection or patient comfort.

Preferably, the control unit is configured to operate the field generator to generate the positioning field over a time period of less than about one second. Such comparably provision of the positioning field allows for an identification of a reaction caused by the positioning field but, at the same time, preventing or minimizing further reaction of the muscular structure which may disturb the patient or cause undesired effects.

Preferably, the control unit is configured to operate the field generator to generate the positioning field with characteristic properties. Such characteristic properties can be or comprise a predefined variation of the intensity of the field or a shape of the field. By providing the positioning field with specific characteristics or a predefined pattern allows for efficiently identifying a feedback or reaction of the muscular structure correlating to the positioning field. Thereby, an expected time of feedback or reaction can be involved with additionally allows for accurately correlating the feedback or reaction to the provision of the positioning field. Further, the correlation between the positioning field pattern and the feedback pattern can be a measure for qualifying appropriateness of the position of the field generator.

Preferably, the stimulation arrangement comprises a sensor unit in communication with the control unit, wherein the sensor unit is configured to be positioned at the patient to sense the feedback from the muscular structure of the patient and to provide a feedback signal representing the sensed feedback, and the control unit is configured to obtain the feedback signal from the sensor unit.

Similarly as in connection with the field generator, the term “positioned at” as used in connection with the sensor unit can relate to the sensor unit or a part thereof being physically in contact with the body of the patient or distant to it. It may also involve being at least partially located inside the body such as, e.g., inside an oral cavity or the like. In order to be configured for being positioned at an appropriate location, a sensor unit can be formed to be suited to the location. Also, it can be equipped with an appropriate mounting structure for being secured at the location.

The term “sensing a feedback from the muscular system” as used herein relates to any feedback signal allowing to identify activation of the muscular system. In particular, such feedback can be a feedback directly identified at or from the muscular system such as, e.g., a contraction of one or more muscles of the muscular system. Also, such feedback can be a feedback indirectly associated to activation of the muscular system. For example, the feedback can be a feedback of a respiratory system of the patient. More specifically, in connection with activation of aspiration muscular structure, such as activation of the diaphragm by either being directly stimulated by the field generator or indirectly via stimulation of the phrenic nerve(s), the feedback can be a flow sensed in the mouth of the patient or elsewhere in the respiratory system.

The sensor unit may comprise a single sensor or a group of sensors. In connection with ventilation, the sensor unit may comprise an airway pressure or flow sensor, and the feedback signal may have an airway pressure or flow component. Alternatively or additionally, the sensor unit can comprise an esophageal pressure sensor and the feedback signal has an esophageal pressure component. Further alternatively or additionally, the sensor unit may have a belt, e.g. an abdominal belt, to sense movement of the muscular structure such as expansion and/or retraction of the diaphragm. Still further alternatively or additionally, the sensor unit may comprise an electrode configured to determine an activity of the muscular structure and the feedback signal has an electrode component. Other possible sensors may be electromyography (EMG) sensors, oesophagus catheters or accelerometers. In addition to one or more sensors, the sensor unit can further include other means such as a communication adapter, a mounting arrangement or similar structures.

Provision of the feedback signal by the sensor unit can include forwarding of the feedback signal to a target such as the control unit (push), or a making available of the feedback signal to be collected or gathered by the control unit (pull). Likewise, obtaining the feedback signal by the control unit can be a receiving of the feedback signal forwarded by the senor unit or a collecting or gathering of the feedback signal from the sensor unit.

Preferably, the control unit is configured to identify in the obtained feedback signal a characteristic feedback from the muscular structure of the patient induced by the characteristic properties of the positioning field. By such identification of the characteristic feedback, the feedback can efficiently be correlated to the positioning field.

In connection with the sensor unit, the control unit preferably is configured to store the obtained feedback signal. For being configured to store the obtained feedback signal, the control unit can be equipped with any suitable volatile or permanent data storage structure such as hard disk, a memory chip, a RAM, any combination thereof, or the like. Alternatively or additionally, it may be connected to an external data storage. Such storage of feedback signals allows for making conveniently available plural feedback signals for evaluation. For example, feedback signals induced by applying the field generator at different locations can be compared and the position effecting the most appropriate feedback signal can be chosen for therapy. Like this, the appropriate or best position can particularly efficiently be found in an (semi-) automatic manner. Thus, the control unit preferably is configured to compare stored feedback signals and to choose one of the stored feedback signals.

The control unit preferably is configured to compare feedback signal intensities of the stored feedback signals when choosing the one of the stored feedback signals. Like this, a position providing particularly appropriate feedback can be selected. Specifically, the control unit preferably is configured to choosing the one of the stored feedback signals having the highest feedback signal intensity. The position of the field generator at the patient associated to the feedback signal having the highest feedback signal intensity may be identified as the position having the highest stimulation efficiency. Thus, this position may be considered as the best position evaluated such that field generator can efficiently be placed at the best position.

The control unit preferably is configured to predefine a target feedback signal intensity and/or a target feedback signal characteristics, to determine a response time being the time from start of generation of the positioning field to reach of the target feedback signal intensity and/or the target feedback signal characteristics for each of the stored feedback signals and to compare the determined response times when choosing the one of the stored feedback signals. By such configuration response times of different positions of the field generator can be involved when selecting the appropriate position of the field generator.

Specifically, the control unit preferably is configured to choosing the one of the stored feedback signals having the shortest response time. Particularly, in combination with the increasing intensity of the positing field this may efficiently be implemented. The position of the field generator at the patient associated to the feedback signal having the shortest response time may be the position having the highest stimulation efficiency. Thus, this position may be the best position evaluated such that field generator can efficiently be placed at the best position.

Further, the control unit preferably is configured to stop operation of the field generator to generate the positioning field when the predefined target feedback signal intensity and/or the target feedback signal characteristics is achieved. Like this, activation of the muscular structure may be stopped or prevented after the feedback is received. Thus, unnecessary activation or over-activation and involved discomfort can be prevented or lowered.

Preferably, the positioning field has a linearly increasing intensity. Such linear increase allows for adapting sensitivity of the patient to the stimulation such that a higher intensity can be applied without inappropriately affecting the patient.

Preferably, the control unit is configured to operate the field generator to generate the positioning field during an end-expiratory window of a breathing cycle of the patient. The end-expiration window typically is a section of the breathing cycle, where flow is close to zero or pressure is constant. Such generation of the positioning field allows for an efficient identification of a feedback from the patient, particularly, when using an airway flow or pressure sensor.

Preferably, the positioning field has a first portion with the increasing intensity and a second portion with a decreasing or constant intensity, wherein the first portion of the positioning field has a first temporal width and the second portion of the positioning field has a second temporal width being considerably smaller than the first temporal width. Such ramp stimulation signal, allows for a comfortable stimulation.

Thereby, the first temporal width preferably is at least four times the second temporal width, advantageously at least eight times the second temporal width and more advantageously at least fifteen times the second temporal width.

Alternatively, the second temporal width preferably is about 0 seconds. Like this, sudden termination of application the positioning field and, thus, activation of the muscular structure can be achieved.

Preferably, the positioning field is a field train. Thereby, the field train is either continuously generated or, preferably, in sequences of pulses comparably quickly following each other. Such train may achieve to stimulate a nerve or muscle such that a tetanic contraction or activation is induced.

Advantageously, the train is provided by increasing the intensity (field strength) and/or frequency until a target intensity and frequency is achieved (ramp protocol). Like this, sudden convulsion or discomfort can be decreased or minimized. All of these parameters are summarized under the term “temporal characteristics” or “temporal parameters” of the spatial field. These temporal parameters can be adjusted manually via an input interface or be controlled automatically by an adjustment mechanism or the control unit.

The parameters of the voltage or current waveform applied to generate the spatial field may affect the temporal characteristics of the spatial field, including pulse shape, amplitude, width, polarity, and repetition frequency; duration of and interval between bursts or trains of pulses; total number of pulses; and interval between stimulation sessions and total number of sessions have, amongst others, an influence on the field strength and determine if and with which intensity or “dose” a target area or target tissue can be stimulated.

The temporal characteristics and spatial distribution of the spatial field can be tuned in such a way that the desired activation (activation feedback) of the muscular structure is achieved. Thereby, the activation feedback (signal) may refer to a signal that indicates appropriate characteristics of muscular structure activation, e.g. a signal that reaches or exceeds a target value (threshold), a signal that exhibits a certain curve pattern or shape, a signal that fulfils a certain algorithm known to represent appropriate target muscular structure activation in the desired strength, or any combination thereof. The activation feedback (signal) may comprise a feedback in particular about a desired muscle activation strength that shall be reached before the adjustment mechanism stops variation. The appropriate activation feedback signal characteristics can for example be defined by a user via an input interface or be detected by algorithms.

Thereby, the field train preferably comprises a sequence of pulses of the spatial field. The term “pulses” in connection with the spatial field relates to a plurality of pulses of generation of the spatial field over a comparably short time and with a comparably short interruption between two subsequent pulses. In contrast, a single pulse relates to the generation of the spatial field over a comparably short time and with a comparably long interruption between two subsequent pulses. Typically, single pulses are provided at frequencies lower than 10 Hertz (Hz) such as, e.g. at 5 Hz or below, or single pulses are initiated by the user or practitioner. The pulses of the field train can have a temporal width of about 10 microseconds (μs) to about 300 μs. Such pulses can activate nerves and muscle structure and are identifiable by the patient or by a sensor. In particular, such single pulses may cause a single convulsion of a muscle or muscular structure.

Thereby, the sequence of pulses preferably comprises a frequency in a range of about 15 Hz to about 30 Hz. Such frequency allows for achieving that perception of the train is more or less continuous.

Preferably, the control unit is configured to operate the field generator to generate a regular stimulation field to therapeutically activate the muscular structure of the patient, wherein a maximum intensity of the positioning field is about 40% of a maximum intensity of the regular stimulation field, advantageously about 30% of the maximum intensity of the regular stimulation field, or more advantageously about 20% of the maximum intensity of the regular stimulation field. The therapeutic activation of the muscular system can be any target or aimed activation of the muscular structure via direct or indirect stimulation. For example, it can be an activation of the aspiration muscular structure to induce or assist breathing.

By dimensioning the intensity of the positioning field essentially below the intensity of the regular stimulation field, it can be prevented or limited that a therapeutic or other perceivable effect is induced when positioning the field generator. In particular, the positioning field can be dimensioned to allow just sensing of the feedback signal but not starting any considerable therapeutic effect. For example, in connection with ventilation, the positioning field can be dimensioned such that no ventilation of ventilation cycle is induced but that the aspiration muscular system is only activated to an extent sufficient to generate the feedback signal.

Thereby, the positioning field preferably has characteristics different from characteristics of the regular stimulation field. Besides intensity, the characteristics may comprise change in or course of intensity, temporal occurrence, temporal width, or the like. By having specific characteristics, the positioning field may be efficiently identified and differed from the regular stimulation field.

Preferably, the stimulation device comprises a user interface having a trigger in communication with the control unit, wherein the control unit is configured to operate the field generator to generate the positioning field upon activation of the trigger. The trigger may be implemented as physical switch or button, or as a graphical representation on a touch sensitive screen. Such trigger allows for manually activating the positioning field. Like this, a physician may provide the positioning field and immediately check for the feedback.

In another aspect, the invention is a method of identifying a position of a field generator at a human or animal patient to activate a muscular structure of the patient by a spatial field generated by the field generator. The method comprises the steps of (i) arranging the field generator at the patient; (ii) operating the field generator by increasing an intensity of the spatial field; (iii) identifying a feedback of the muscular system of the patient; and (iv) defining the position of the field generator at the patient based on the identified feedback of the muscular structure.

The method according to the invention and its preferred embodiments described below allows for achieving the effects and benefits of the stimulation device and its preferred embodiments described above.

The feedback of the muscular system can be identified by a practitioner observing the patient and recognizing a reaction related to the operation of the field generator. Preferably, identifying a feedback of the muscular system of the patient comprises sensing the feedback of the muscular structure. Such sensing allows for precisely recognizing and dimensioning the feedback.

Preferably, the field generator is operated such that the feedback of the muscular system of the patient lasts less than about 0.5 second, preferably less than about 0.2 seconds. Such comparably short stimulation may be sufficient for efficiently positioning.

The method preferable comprises a step of comparing plural feedbacks of the muscular system each obtained from a different position of the field generator at the patient, wherein defining the position of the field generator at the patient comprises identifying one of the plural feedbacks of the muscular system as appropriate feedback. Such comparison and identification allow for efficiently choosing a particularly appropriate position. Thus, defining the position of the field generator at the patient preferably comprises choosing the position associated to the appropriate feedback.

Preferably, comparing the plural feedbacks of the muscular system comprises comparing intensities of the feedbacks of the muscular system and identifying the one of the plural feedbacks of the muscular system as the appropriate feedback comprises choosing the feedback having the highest intensity. The maximum intensity of the feedback may be indicative for stimulation efficiency. Thus, selecting the position inducing the highest feedback intensity may achieve to efficiently identify the most or a highly appropriate position of the field generator.

Alternatively or additionally, the method preferably comprises a step of predefining a target feedback intensity, determining a response time being the time from start of generation of the positioning field to reach of the target feedback intensity for each of the plural feedbacks and comparing the determined response times when identifying the one of the plural feedbacks of the muscular system as the appropriate feedback. The time to achieve a certain feedback intensity may also be indicative for stimulation efficiency.

Specifically, the method preferably comprises a step of choosing the one of the feedbacks of the muscular system having the shortest response time. Thus, selecting the position inducing the shorted response time may achieve to efficiently identify the most or a highly appropriate position of the field generator.

The method preferably comprises a step of stopping operation of the field generator to generate the positioning field when the predefined target feedback is achieved. Like this, stimulation unnecessary for finding the appropriate position of the field generator can be prevented.

Preferably, the positioning field has a linearly increasing intensity.

Preferably, the positioning field has a first portion with the increasing intensity and a second portion with a decreasing intensity, wherein the first portion of the positioning field has a first temporal width and the second portion of the positioning field has a second temporal width being considerably smaller than the first temporal width.

Thereby, the first temporal width preferably is at least four times the second temporal width, advantageously at least eight times the second temporal width and more advantageously at least fifteen times the second temporal width.

Alternatively, the second temporal width preferably is about 0 seconds.

Preferably, the positioning field is a field train. Thereby, the field train preferably comprises a sequence of pulses of the spatial field. The sequence of pulses preferably comprises a frequency in a range of about 15 Hz to about 30 Hz.

Preferably, a maximum intensity of the positioning field is about 40% of a maximum intensity of a regular stimulation field, advantageously about 30% of the maximum intensity of the regular stimulation field, or more advantageously about 20% of the maximum intensity of the regular stimulation field, wherein the regular stimulation field is configured to therapeutically activate the muscular structure of the patient.

Thereby, the positioning field preferably has characteristics different from characteristics of the regular stimulation field.

Preferably, the method comprises a step of manually triggering the field generator to generate the positioning field.

Preferably, when operating the field generator, a nervous system of the patient and, preferably a phrenic nerve or a respiratory nerve of the patient, is stimulated. Such stimulation allows for a convenient indirect activation of the muscular system of the patient.

Preferably, the field generator is operated to generate a positioning field with characteristic properties. Thereby, identifying a feedback of the muscular system of the patient preferably comprises identifying an activity of the muscular system correlating to the characteristic properties of the positioning field. The characteristic properties of the positioning field, such as a predefined varying intensity, allows for an efficient and reliant identification of the feedback which typically correlates to the field.

Preferably, the feedback of the muscular system of the patient is identified by sensing a flow and/or a pressure. Particularly, flow or pressure may by an airway flow or airway pressure of the patient. Thereby, the field generator preferably is operated during an end-expiratory window of a breathing cycle of the patient

Preferably, the method is performed with a stimulation device as described above. Like this, the method can efficiently be implemented, e.g. in a fully or partially automated manner.

### BRIEF DESCRIPTION OF THE DRAWINGS

The stimulation arrangement according to the invention and the method according to the invention are described in more detail hereinbelow by way of exemplary embodiments and with reference to the attached drawings, in which:

FIG. 1 shows a schematic view of an embodiment of a stimulation arrangement according to the invention implementing an embodiment of a method according to the invention;

FIG. 2 shows graphs of field and feedback intensities of a first mode of finding an appropriate position of a field generator of the stimulation arrangement of FIG. 1; and

FIG. 3 shows graphs of field and feedback intensities of a second mode of finding an appropriate position of the field generator of the stimulation arrangement of FIG. 1.

### DESCRIPTION OF EMBODIMENTS

In the following description certain terms may be used for reasons of convenience and are not intended to limit the invention. The terms “right”, “left”, “up”, “down”, “under” and “above” refer to directions in the figures. The terminology comprises the explicitly mentioned terms as well as their derivations and terms with a similar meaning. Also, spatially relative terms, such as “beneath”, “below”, “lower”, “above”, “upper”, “proximal”, “distal”, and the like, may be used to describe one element's or feature's relationship to another element or feature as illustrated in the figures. These spatially relative terms are intended to encompass different positions and orientations of the devices in use or operation in addition to the position and orientation shown in the figures. For example, if a device in the figures is turned over, elements described as “below” or “beneath” other elements or features would then be “above” or “over” the other elements or features. Thus, the exemplary term “below” can encompass both positions and orientations of above and below. The devices may be otherwise oriented (rotated 90 degrees or at other orientations), and the spatially relative descriptors used herein interpreted accordingly. Likewise, descriptions of movement along and around various axes include various special device positions and orientations.

To avoid repetition in the figures and the descriptions of the various aspects and illustrative embodiments, it should be understood that many features are common to many aspects and embodiments. Omission of an aspect from a description or figure does not imply that the aspect is missing from embodiments that incorporate that aspect. Instead, the aspect may have been omitted for clarity and to avoid prolix description. In this context, the following applies to the rest of this description: If, in order to clarify the drawings, a figure contains reference signs which are not explained in the directly associated part of the description, then it is referred to previous or following description sections. Further, for reason of lucidity, if in a drawing not all features of a part are provided with reference signs it is referred to other drawings showing the same part. Like numbers in two or more figures represent the same or similar elements.

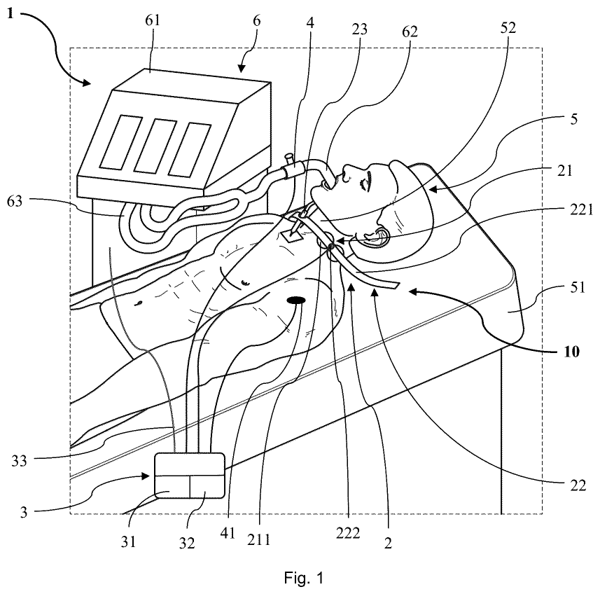

FIG. 1 shows a ventilation arrangement 1 comprising a ventilation machine 6 and an embodiment of a stimulation arrangement 10 according to the invention. The stimulation arrangement 10 has an electro-magnetic induction device 2 (in the following also referred to as EMI device), a control unit 3 and a sensor unit 4.

The EMI device 2 comprises an electro-magnetic field generator 21 with two coils 211 as coil design. The coils 211 are located in one common plane and configured to generate a spatial electro-magnetic field. The electro-magnetic field has a central targeted shape with a focality area at which the electro-magnetic field maximally extends. The EMI device 2 has a mounting arrangement 22 with a neck arc 221 arranged at a neck 52 of a patient 5 and fixed to a bed 51 the patient 5 lies on. The neck arc 221 is equipped with a joint 222 as repositioning structure of the EMI device 2. The joint 222 holds the coils 211 at the neck 52 of the patient 5. When operated, the two coils 211 generate the electro-magnetic field towards the neck 52 of the patient 5, wherein due to its targeted shape the focality area maximally extends into the neck 52.

The ventilation machine 6 comprises a ventilator 61 as air flow generator from which ventilation tubes 63 extend, and a mouthpiece 62 as conduit interface. The mouthpiece 62 is a tube provided through a mouth of the patient into the respiratory system of the patient 5.

The sensor unit 4 comprises a flow sensor arranged between the mouthpiece 62 and the tubes 63, and an abdominal belt as diaphragm contraction sensor. The control unit 3 is configured to receive sensor signals provided by the sensor unit 4.

The control unit 3 has a user interface 31 for exchanging information with a practitioner supervising or setting up mechanical and electro-magnetically stimulated ventilation of the patient 5. Specifically, the user interface 31 is embodied as touch screen allowing to in- and output information. Further, the control unit 3 is equipped with a device interface 32 arranged to be coupled to an interface unit of the ventilation machine 6, the EMI device 2 and the sensor unit 4 by wires 33. Like this, the control unit 3 is in communication with the ventilation machine 6, the EMI device 2 and the sensor unit 4.

More specifically, the control unit 3 is configured to receive ventilation data about the ventilation of the patient 5 from the ventilation machine 6 and to control the EMI device 2 to generate the electro-magnetic field in accordance with the evaluated ventilation data. Furthermore, the control unit 3 is configured to manipulate the joint 222 to automatically vary the position of the focality area 213 of the electro-magnetic field generated by the coils 211 and to vary the field strength of the electro-magnetic field. The aim of varying field strength and position of the electro-magnetic field 212 is to adjust the electro-magnetic field such that it specifically optimally stimulates a phrenic nerve of the patient 5. Upon stimulation of the phrenic nerve, a diaphragm as muscular structure of the patient 5 is activated. Thereby, aspiration is induced.

The ventilation machine 6 is configured to mechanically ventilate the patient 5 by advancing air through the mouthpiece 62 into the respiratory system of the patient 5. More specifically, the ventilator 61 is configured to deliver the air through the mouthpiece 62. The control unit 3 is configured to control the ventilator 61 to deliver the air according to a breathing scheme defined in the control unit 3. Moreover, the control unit 3 regulates the activation of the diaphragm in coordination with the breathing scheme such that activation of the diaphragm via the phrenic nerve is coordinated with the mechanical ventilation of the patient 5. Specifically, for such therapeutical activation of the diaphragm in coordination with the mechanical ventilation, the control unit 3 is configured to operate the field generator 21 such that the coils 211 generate a regular stimulation field. This regular stimulation field stimulates the phrenic nerve which activates the diaphragm.

For being able to provide various treatments during ventilation, the control unit 3 is configured to define combinations of a stimulation duration and a repetition rate, and to operate the EMI device 2 in accordance with the defined stimulation duration and the determined repetition rate. Thereby, the control unit 3 provides a selection of treatments to the practitioner via the user interface 31. The practitioner selects an appropriate treatment and sets parameters involved.

When setting up the EMI device 2, the field generator 21 and particularly its coils 211 have to be appropriately positioned. More specifically, the coils 211 have to be located and oriented such that the electro-magnetic field optimally reach the phrenic nerve. For such positioning of the coils 211, the control unit 3 is configured to operate the EMI device 2 and particularly its field generator 21 to generate a positioning field having an increasing intensity.

The positioning field is a field train comprising a sequence of pulses of electro-magnetic field. Thereby, starting from a zero intensity each pulse has a constantly higher intensity than its preceding pulse such that the field train has a linearly increasing intensity. More specifically, field train has a first portion with the increasing intensity and a second portion with a decreasing intensity, wherein the second portion has a temporal width of 0 seconds. Thus, the positioning field immediately stops after increasing the intensity to a maximum intensity. The maximum intensity of the positioning field is 40%, 30%, or 20% of a maximum intensity of the regular stimulation field or less.

The control unit 3 is configured to store sensor signals received when operating the field generator 21 to generate the positioning field as feedback signals. For finding the appropriate position of the coils 211, the control unit 3 is embodied to evaluate the feedback signals in two modes, wherein the practitioner may select the mode to be applied on the touch screen.

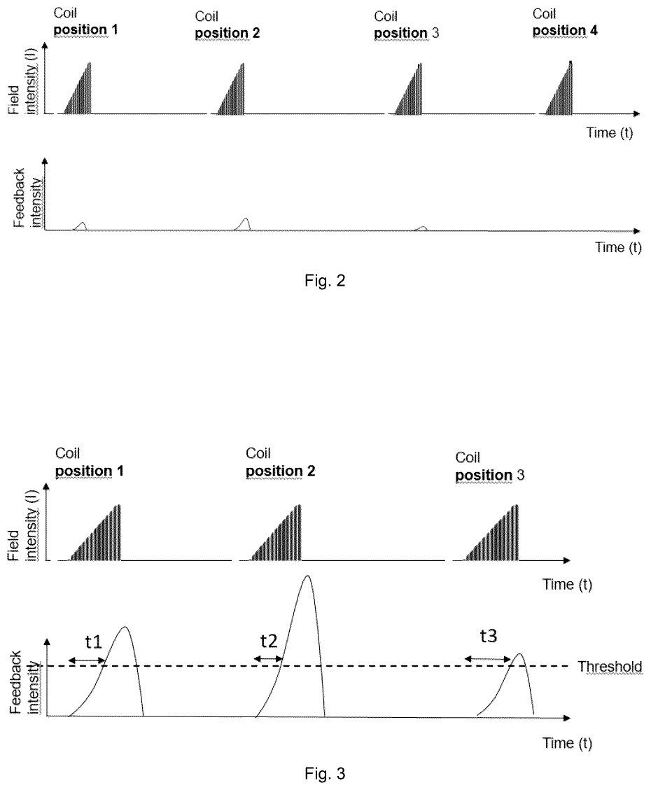

In a first maximum intensity mode shown in FIG. 2, the coils 211 are positioned at various different positions at the neck 52 of the patient 5, i.e., in the example of FIG. 2 this is four different positions. The control unit 3 operates the field generator 21 at each of the four positions to provide the positioning field having an increasing field intensity (I). Further, the control unit 3 receives and stores the feedback signals of all four positions. Then, it compares signal intensities of the stored feedback signals and chooses the one of the stored feedback signals having the highest feedback signal intensity. In the example of FIG. 2 this is the feedback signal associated to position 2. Thus, the control unit 3 identifies position 2 as the best of the analysed four positions and provides this information to the practitioner.

In a second shortest time to response mode shown in FIG. 3, the coils 211 are also positioned at various different positions at the neck 52 of the patient 5, i.e., in the example of FIG. 3 this is three different positions. The control unit 3 operates the field generator 21 at each of the three positions to provide the positioning field as well as receives and stores the feedback signals of all three positions. Furthermore, the control unit 3 predefines a threshold as target feedback signal intensity, wherein the practitioner may input such threshold via the user interface 31. The control unit 3 then determines a response time being the time from start of generation of the positioning field to reach of the threshold for each of the stored feedback signals. Further, it compares the determined response times and chooses the one of the stored feedback signals having the shortest response time. In the example of FIG. 3 this is the feedback signal associated to position 2. Thus, the control unit 3 identifies position 2 as the best of the analysed three positions and provides this information to the practitioner.

This description and the accompanying drawings that illustrate aspects and embodiments of the present invention should not be taken as limiting-the claims defining the protected invention. In other words, while the invention has been illustrated and described in detail in the drawings and foregoing description, such illustration and description are to be considered illustrative or exemplary and not restrictive. Various mechanical, compositional, structural, electrical, and operational changes may be made without departing from the spirit and scope of this description and the claims. In some instances, well-known circuits, structures and techniques have not been shown in detail in order not to obscure the invention. Thus, it will be understood that changes and modifications may be made by those of ordinary skill within the scope and spirit of the following claims. In particular, the present invention covers further embodiments with any combination of features from different embodiments described above and below. For example, in an advantageous variant of the second shortest time to response mode the control unit stops operation of the field generator when the threshold is achieved. Like this, impact on the patient can be reduced.

The disclosure also covers all further features shown in the FIGS. individually although they may not have been described in the afore or following description. Also, single alternatives of the embodiments described in the figures and the description and single alternatives of features thereof can be disclaimed from the subject matter of the invention or from disclosed subject matter. The disclosure comprises subject matter consisting of the features defined in the claims or the exemplary embodiments as well as subject matter comprising said features.

Furthermore, in the claims the word “comprising” does not exclude other elements or steps, and the indefinite article “a” or “an” does not exclude a plurality. A single unit or step may fulfil the functions of several features recited in the claims. The mere fact that certain measures are recited in mutually different dependent claims does not indicate that a combination of these measures cannot be used to advantage. The terms “essentially”, “about”, “approximately” and the like in connection with an attribute or a value particularly also define exactly the attribute or exactly the value, respectively. The term “about” in the context of a given numerate value or range refers to a value or range that is, e.g., within 20%, within 10%, within 5%, or within 2% of the given value or range. Components described as coupled or connected may be electrically or mechanically directly coupled, or they may be indirectly coupled via one or more intermediate components. Any reference signs in the claims should not be construed as limiting the scope.

1. A stimulation arrangement comprising:
a field generator configured to generate a spatial field, wherein the field generator preferably comprises a coil and a magnetic stimulator connected to the coil and
a control unit in communication with the field generator,
wherein the field generator is configured to be positioned at a human or animal patient such that a muscular structure of the patient is activatable by the spatial field,
wherein the control unit is configured to control the field generator to generate the spatial field, and
wherein the control unit is configured to operate the field generator to generate a positioning field having an increasing intensity.

2. The stimulation arrangement of claim 1, wherein the control unit is configured to operate the field generator to generate the positioning field over a time period of less than about one second, and/or to operate the field generator to generate the positioning field with characteristic properties.

3. (canceled)

4. The stimulation arrangement of claim 1, comprising a sensor unit in communication with the control unit, wherein
the sensor unit is configured to be positioned at the patient to sense the feedback from the muscular structure of the patient and to provide a feedback signal representing the sensed feedback, and
the control unit is configured to obtain the feedback signal from the sensor unit.

5. The stimulation arrangement of claim 2, wherein the control unit is configured to operate the field generator to generate the positioning field with characteristic properties and to identify in the obtained feedback signal a characteristic feedback from the muscular structure of the patient induced by the characteristic properties of the positioning field.

6. The stimulation arrangement of claim 4, wherein the control unit is configured to store the obtained feedback signal,
wherein the control unit preferably is configured to compare stored feedback signals and to choose one of the stored feedback signals,
wherein the control unit preferably is configured to compare feedback signal intensities of the stored feedback signals when choosing the one of the stored feedback signals, and
wherein the control unit preferably is configured to choosing the one of the stored feedback signals having the highest feedback signal intensity.

7-9. (canceled)

10. The stimulation arrangement of claim 6, wherein the control unit is configured
to predefine a target feedback signal intensity, to determine a response time being the time from start of generation of the positioning field to reach of the target feedback signal intensity for each of the stored feedback signals and to compare the determined response times when choosing the one of the stored feedback signals,
and/or to predefine a target feedback signal characteristics, to determine a response time being the time from start of generation of the positioning field to reach of the target feedback signal characteristics for each of the stored feedback signals and to compare the determined response times when choosing the one of the stored feedback signals.

11. (canceled)

12. The stimulation arrangement of claim 10, wherein the control unit is configured
to choosing the one of the stored feedback signals having the shortest response time, and/or
to stop operation of the field generator to generate the positioning field when the predefined target feedback signal intensity and/or the target feedback signal characteristics is achieved.

13. (canceled)

14. The stimulation arrangement of claim 4, wherein the sensor unit comprises an airway flow sensor and/or an airway pressure sensor, wherein the control unit preferably is configured to operate the field generator to generate the positioning field during an end-expiratory window of a breathing cycle of the patient.

15. (canceled)

16. The stimulation arrangement of claim 1, wherein the positioning field has
a linearly increasing intensity, and/or
a first portion with the increasing intensity and a second portion with a decreasing intensity, wherein the first portion of the positioning field has a first temporal width and the second portion of the positioning field has a second temporal width being considerably smaller than the first temporal width, and/or
a first portion with the increasing intensity and a second portion with a constant intensity, wherein the first portion of the positioning field has a first temporal width and the second portion of the positioning field has a second temporal width being considerably smaller than the first temporal width,
wherein the first temporal width preferably is at least four times the second temporal width, advantageously at least eight times the second temporal width and more advantageously at least fifteen times the second temporal width,
wherein the second temporal width preferably is about 0 seconds.

17-20. (canceled)

21. The stimulation arrangement of claim 1, wherein the positioning field is a field train,
wherein the field train preferably comprises a sequence of pulses of the spatial field,
wherein the sequence of pulses preferably comprises a frequency in a range of about 15 Hz to about 30 Hz.

22-23. (canceled)

24. The stimulation arrangement of claim 1, wherein the control unit is configured to operate the field generator to generate a regular stimulation field to therapeutically activate the muscular structure of the patient, wherein a maximum intensity of the positioning field is about 40% of a maximum intensity of the regular stimulation field, advantageously about 30% of the maximum intensity of the regular stimulation field, or more advantageously about 20% of the maximum intensity of the regular stimulation field, wherein the positioning field preferably has characteristics different from characteristics of the regular stimulation field.

25. (canceled)

26. The stimulation arrangement of claim 1, comprising a user interface having a trigger in communication with the control unit, wherein the control unit is configured to operate the field generator to generate the positioning field upon activation of the trigger.

27. (canceled)

28. A method of identifying a position of a field generator at a human or animal patient to activate a muscular structure of the patient by a spatial field generated by the field generator, preferably performed with a stimulation device of claim 1, comprising
arranging the field generator at the patient,
operating the field generator by increasing an intensity of the spatial field,
identifying a feedback of the muscular system of the patient, and
defining the position of the field generator at the patient based on the identified feedback of the muscular structure.

29. The method of claim 28, wherein the field generator is operated such that the feedback of the muscular system of the patient lasts less than about 0.5 second, preferably less than about 0.2 seconds, wherein identifying a feedback of the muscular system of the patient preferably comprises sensing the feedback of the muscular structure.

30. (canceled)

31. The method of claim 29, comprising a step of comparing plural feedbacks of the muscular system each obtained from a different position of the field generator at the patient, wherein defining the position of the field generator at the patient comprises identifying one of the plural feedbacks of the muscular system as appropriate feedback, wherein defining the position of the field generator at the patient preferably comprises choosing the position associated to the appropriate feedback.

32. (canceled)

33. The method of claim 31,
wherein comparing the plural feedbacks of the muscular system comprises comparing intensities of the feedbacks of the muscular system, and identifying the one of the plural feedbacks of the muscular system as the appropriate feedback comprises choosing the feedback having the highest intensity, and/or
wherein the method comprises
predefining a target feedback intensity,
determining a response time being the time from start of generation of the positioning field to reach of the target feedback intensity for each of the plural feedbacks, and
comparing the determined response times when identifying the one of the plural feedbacks of the muscular system as the appropriate feedback,
wherein the method preferably comprises a step of choosing the one of the feedbacks of the muscular system having the shortest response time, and/or a step of operation of the field generator to generate the positioning field when the predefined target feedback is achieved.

34-36. (canceled)

37. The method of claim 28, wherein the positioning field has
a linearly increasing intensity, and/or
a first portion with the increasing intensity and a second portion with a decreasing intensity,
wherein the first portion of the positioning field has a first temporal width and the second portion of the positioning field has a second temporal width being considerably smaller than the first temporal width,
wherein the first temporal width preferably is at least four times the second temporal width, advantageously at least eight times the second temporal width and more advantageously at least fifteen times the second temporal width, and
wherein the second temporal width preferably is about 0 seconds.

38-40. (canceled)

41. The method of claim 28, wherein the positioning field is a field train,
wherein the field train preferably comprises a sequence of pulses of the spatial field,
wherein the sequence of pulses preferably comprises a frequency in a range of about 15 Hz to about 30 Hz.

42-43. (canceled)

44. The method of claim 28, wherein a maximum intensity of the positioning field is about 40% of a maximum intensity of a regular stimulation field, advantageously about 30% of the maximum intensity of the regular stimulation field, or more advantageously about 20% of the maximum intensity of the regular stimulation field, wherein the regular stimulation field is configured to therapeutically activate the muscular structure of the patient, wherein the positioning field preferably has characteristics different from characteristics of the regular stimulation field.

45. (canceled)

46. The method of claim 28,
wherein the method comprises a step of manually triggering the field generator to generate the positioning field, and/or
wherein operating the field generator stimulates a nervous system of the patient and, preferably a phrenic nerve or a respiratory nerve of the patient, and/or
wherein the field generator is operated to generate a positioning field with characteristic properties, and/or
wherein identifying a feedback of the muscular system of the patient comprises identifying an activity of the muscular system correlating to the characteristic properties of the positioning field, and/or
wherein the feedback of the muscular system of the patient is identified by sensing a flow and/or pressure, wherein the field generator preferably is operated during an end-expiratory window of a breathing cycle of the patient.

47-52. (canceled)
